(19) United States
(12) Patent Application Publication
(10) Pub. No.: US 2022/0193165 A1
(43) Pub. Date: Jun. 23, 2022

(54) Oral Cannabis Substance and Method of Making Such Substance

(71) Applicant: Venadi LLC, Houston, TX (US)

(72) Inventor: Jacob Spencer Rives, Houston, TX (US)

(21) Appl. No.: 17/557,250

(22) Filed: Dec. 21, 2021

Related U.S. Application Data
(60) Provisional application No. 63/128,556, filed on Dec. 21, 2020.

(51) Int. Cl. A61K 36/185; A61K 31/465; A61K 9/00; A61K 47/46

(52) U.S. Cl. CPC A61K 36/185; A61K 31/465; A61K 9/0056; A61K 47/46; A61K 2236/15; A61K 2236/53; A61K 2236/37; A61K 2236/31; A61K 2236/333

(57) ABSTRACT

Embodiments provide an orally administered substance that contains cannabis and nicotine for medicinal and recreational purposes.

### CROSS-REFERENCE TO RELATED APPLICATIONS

The current application claims priority to United States Provisional Application 63/128,556 filed Dec. 21, 2020, the entirety of which is incorporated by reference.

### FIELD OF THE DISCLOSURE

Aspects of the disclosure relate to orally administered substances, such as chewing compositions. More specifically, aspects of the disclosure relate to cannabis and a nontobacco-based nicotine chewing compositions.

### BACKGROUND

Chewing substances have been administered for both medicinal and recreation activities for many years. Such substances are recreationally used by millions of individuals on a daily basis. These individuals enjoy the taste, texture and physical effects provided by the substance. Conventional chewing substances, such as chewing tobacco, are known. In order to provide a greater appeal for the product, manufacturers provide different flavoring to please user pleasure.

Cannabis and materials related to cannabis, such as cannabinoids, have been used in various capacities in the medical field. Cannabis has been used to treat anxiety as well as pain relief. Cannabis has also been identified as an anti-inflammatory material that can help inflammation from a chronic or post-operative condition. Use of cannabis and cannabis products have been avoided for many years due to prohibitions on such substances. Attitudes have changed, however, for cannabis products as legal constraints have been loosened and the benefits of cannabis can be explored.

Research into cannabis has taken on greater importance as laws related to cannabis use and distribution have been modified. To date, however, there are few alternatives to inhaled cannabis administration. There is a need, therefore, to provide an easily administered cannabis substance to an individual in a smokeless/vaporless form. While cannabis liquid forms are known, transport of such liquid forms is difficult for the average individual. Such liquid forms are contained by bottles that fracture or leak, raising concerns about transportation.

There is a further need to provide a conveniently transported cannabis substance that does not have the drawbacks of vapor generated cannabis products, so that the substance can be used without distribution of the substance to others.

There is a need to provide a substance that is easy to package and transport for the average individual.

There is a further need to provide an orally administered substance that incorporates nicotine such that individuals who prefer nicotine based substances may enjoy the beneficial aspects of cannabis.

There is a further need to provide a method of making such substances that is economical and that may be distributed to individuals for use.

There is a further need to provide an oral cannabis product that is easy to transport for the average user.

There is a still further need to provide an oral cannabis product that economical to produce for manufacturers as well as the purchasing public.

### SUMMARY

So that the manner in which the above recited features of the present disclosure can be understood in detail, a more particular description of the disclosure, briefly summarized below, may be had by reference to embodiments, some of which are illustrated in the drawings. It is to be noted that the drawings illustrate only typical embodiments of this disclosure and are therefore not to be considered limiting of its scope, for the disclosure may admit to other equally effective embodiments without specific recitation. Accordingly, the following summary provides just a few aspects of the description and should not be used to limit the described embodiments to a single concept.

In one example embodiment, a substance is disclosed. The substance may comprise a first part comprising a synthetic nicotine and a second part comprising a cannabis component. The substance may further comprise a matrix, wherein the substance is configured to be one of orally chewed and ingested.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the drawings. It is to be noted, however, that the appended drawings illustrate only typical embodiments of this disclosure and are therefore not be considered limiting of its scope, for the disclosure may admit to other equally effective embodiments.

FIG. 1 is a method of production of a cannabis substance, in one non-limiting example embodiment.

To facilitate understanding, identical reference numerals have been used, where possible, to designate identical elements that are common to the FIGURES (“FIGS”). It is contemplated that elements disclosed in one embodiment may be beneficially utilized on other embodiments without specific recitation.

### DETAILED DESCRIPTION

In the following, reference is made to embodiments of the disclosure. It should be understood, however, that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the following aspects, features, embodiments and advantages are merely illustrative and are not considered elements or limitations of the claims except where explicitly recited in a claim. Likewise, reference to “the disclosure” shall not be construed as a generalization of inventive subject matter disclosed herein and should not be considered to be an element or limitation of the claims except where explicitly recited in a claim.

Although the terms first, second, third, etc., may be used herein to describe various elements, components, regions, layers and/or sections, these elements, components, regions, layers and/or sections should not be limited by these terms. These terms may be only used to distinguish one element, components, region, layer or section from another region, layer or section. Terms such as “first”. “second” and other numerical terms, when used herein, do not imply a sequence or order unless clearly indicated by the context. Thus, a first element, component, region, layer or section discussed herein could be termed a second element, component, region, layer or section without departing from the teachings of the example embodiments.

When an element or layer is referred to as being “on.” “engaged to,” “connected to,” or “coupled to” another element or layer, it may be directly on, engaged, connected, coupled to the other element or layer, or interleaving elements or layers may be present. In contrast, when an element is referred to as being “directly on,” “directly engaged to,” “directly connected to,” or “directly coupled to” another element or layer, there may be no interleaving elements or layers present. Other words used to describe the relationship between elements should be interpreted in a like fashion. As used herein, the term “and/or” includes any and all combinations of one or more of the associated listed terms.

Some embodiments will now be described with reference to the FIGURES. Like elements in the various FIGURES will be referenced with like numbers for consistency. In the following description, numerous details are set forth to provide an understanding of various embodiments and/or features. It will be understood, however, by those skilled in the art, that some embodiments may be practiced without many of these details, and that numerous variations or modifications from the described embodiments are possible. As used herein, the terms “above” and “below”, “up” and “down”, “upper” and “lower”, “upwardly” and “downwardly”, and other like terms indicating relative positions above or below a given point are used in this description to more clearly describe certain embodiments.

In one non-limiting embodiment, a method 100 is illustrated. The method 100 may include using different strains of cannabis plant to vary the strength and properties of the substance. In embodiments, cannabis is grown in a “perpetual harvest cycle” wherein plants are grown at different periods of maturity. At 102, a cutting is taken from a mature cannabis plant. At 104, the cutting is placed in a media, such as a nutrient and water rich gel. At 106, the cutting develops a root structure after immersion and resting within the media. At 108, the cutting is transferred to a prepared nutrient rich soil and subsequently irrigated. At 110, a light cycle for the cutting is moderated, such that the specific plant species flowers. At 112, the flowers and buds are removed and dried. In one example embodiment, the drying process occurs at approximately 55 percent humidity for five weeks. As will be understood, drying times may vary according to the water content of the base material

At 114, after drying, the dried flower/bud structures are processed to remove sticks and other non-cannabinoid structures. Such processing decreases the amount of extraneous material thus increasing the weight percentage of cannabinoid bearing material. Cannabinoids can be chemically removed after this processing step to make a cannabinoid extract in liquid form.

To make the liquid form, at 116, ethanol is mixed with the dried flower structures/leaves. Mixing time can be short, for example three (3) minutes. The ethanol frees the cannabinoid chemicals from the dried flow structures/leaves. At 118, the mixed ethanol/flower structure slurry is passed through a first filtration step. The filtration may be through a paper medium, as a non-limiting embodiment. Filtration may also be performed through a metal apparatus, such as a sieve. As will be understood, any number of filtrations may be accomplished.

At 120, the resulting liquid is heated to remove the ethanol. Heating can take one or several heating steps, resulting in the liquid cannabinoid form.

At 122, the cannabinoid liquid form extract obtained at 120, is then added to a synthetic nicotine based tobacco product to create a combination mixture. Different amounts of cannabinoid extract can be added to provide greater or lesser amounts of cannabinoids to the resultant mixture. Table 1 provides seven (7) different formulations for the chewing substance.

In embodiments, the formulation of a water-soluble solution including CBD and non-tobacco based Nicotine (Tobacco Free Nicotine) are disclosed. In embodiments, nicotine will be fully synthetic with zero parts derived directly or indirectly from the tobacco plant or its constituents. The solution can based in a liquid or dry state and will be a food product for purpose of recreational use. The effective ratio of the primary ingredients. CBD and synthetic nicotine, in each of the formulations includes, but is not limited to the formulations described in TABLE 1.

TABLE 1

| | | Non-Tobacco Based |
|---|---|---|
| | CBD (mg) | Nicotine (mg) |
| Formulation 1 | 1 | 1 |
| Formulation 2 | 2 | 1 |
| Formulation 3 | 3 | 1 |
| Formulation 4 | 4 | 1 |
| Formulation 5 | 5 | 1 |
| Formulation 6 | 6 | 1 |
| Formulation 7 | 10 | 3 |

In embodiments, a final product may take several forms. In one such embodiment, dry formulations may be blended with common food grade ingredients such as natural fibers (possibly coconut or avocado), natural sweetener, possibly stabilizing agent, etc. Other examples of fibers may include citrus fruits, apples, peas, barley and oat bran. In some embodiments, the final product may not need refrigeration to maintain freshness. Administration of the product may be placed directly within the mouth of the individual. In some embodiments, no spitting is required for the juices of the product. In other embodiments, the final product may be placed in a pouch that will allow the juices of the fibers and cannabis to be transferred to an exterior of the pouch. The nicotine used may be in the form of a salt to allow for a timed release of the nicotine.

Such fibers, for example, will embody the matrix for chewing by the individual. In other embodiments, liquid formulations may be added to natural fibers. The natural fibers may be porous, in one non-limiting embodiment, to allow for absorption of the liquids into the fibers. As will be understood, the fibers may be water soluble, in a non-limiting example embodiment.

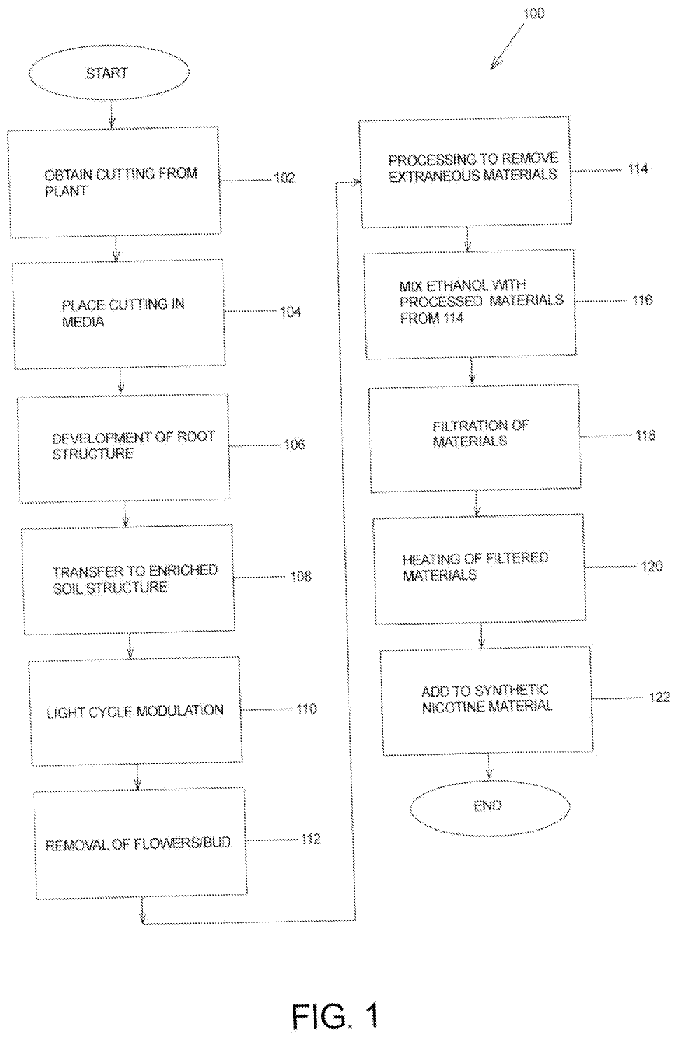

In further embodiments, liquid state materials may added together to formulate a liquid product. In this embodiment, liquid obtained from the method described in relation to FIG. 1, may be added to a liquid state synthetic nicotine. The resulting mixture, in some non-limiting embodiments, may be a water soluble, homogenous solution. This solution is then processed (via spray dry technique) into an inherently stable dry powder state without a reduction in efficacy of the primary ingredients. As the embodiments provided are chewable and not for flammability, the embodiments illustrated provide several advantages over other nicotine products. Other nicotine products are required to be flammable, producing a safety concern. Such safety concerns are eliminated through the embodiments provided herein. Embodiments of the formulations may also be flavored to an extent to allow the product to be used by an increased potential customers, compared to current nicotine based chewables that have limited appeal.

Embodiments of the disclosure provide an easily administered cannabis substance to an individual in a smokeless/vaporless form.

Embodiments provide a conveniently transported cannabis substance that does not have the drawbacks of vapor generated cannabis products, so that the substance can be used without distribution of the substance to others.

Embodiments provide a substance that is easy to package and transport for the average individual.

Embodiments provide an orally administered substance that incorporates synthetic nicotine such that individuals who prefer nicotine based substances may enjoy the beneficial aspects of cannabis and nicotine, without reliance on tabaco products.

Embodiments further provide a method of making such substances that is economical and that may be distributed to individuals for use.

In one example embodiment, a substance is disclosed. The substance may comprise a first part comprising a synthetic nicotine and a second part comprising a cannabis component. The substance may further comprise a matrix, wherein the substance is configured to be one of orally chewed and ingested.

In another example embodiment, the substance may be comprised wherein the matrix includes a natural fiber.

In another example embodiment, the substance may be comprised wherein the natural fiber is a coconut fiber.

In another example embodiment, the substance may be comprised wherein the natural fiber is an avocado fiber.

In another example embodiment, the substance may be comprised wherein the natural fiber is from a citrus fruit.

In another example embodiment, the substance may be comprised wherein the first part is a liquid.

In another example embodiment, the substance may be comprised wherein the first part is a solid.

In another example embodiment, the substance may be comprised wherein the second part is a liquid.

In another example embodiment, the substance may be comprised wherein the substance may be comprised wherein the second part is a solid.

In another example embodiment, the substance may be comprised wherein a weight of the first part is 1 milligram to a second weight of the second part of 1 milligram.

In another example embodiment, the substance may be comprised wherein a weight of the first part is 1 milligram to a second weight of the second part of 2 milligrams.

In another example embodiment, the substance may be comprised wherein a weight of the first part is 1 milligram to a second weight of the second part of 3 milligrams.

In another example embodiment, the substance may be comprised wherein a weight of the first part is 1 milligram to a second weight of the second part of 4 milligrams.

In another example embodiment, the substance may be comprised wherein a weight of the first part is 1 milligram to a second weight of the second part of 5 milligrams.

In another example embodiment, the substance may be comprised wherein a weight of the first part is 1 milligram to a second weight of the second part of 6 milligrams.

In another example embodiment, the substance may be comprised wherein a weight of the first part is 1 milligram to a second weight of the second part of 10 milligrams.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may be varied in many ways. Such variations are not to be regarded as a departure from the disclosure, and all such modifications are intended to be included within the scope of the disclosure.

While embodiments have been described herein, those skilled in the art, having benefit of this disclosure, will appreciate that other embodiments are envisioned that do not depart from the inventive scope. Accordingly, the scope of the present claims or any subsequent claims shall not be unduly limited by the description of the embodiments described herein.

What is claimed is:

1. A substance, comprising:
a first part comprising a synthetic nicotine,
a second part comprising a cannabis component, and
a matrix, wherein the substance is configured to be one of orally chewed and ingested.

2. The substance according to claim 1, wherein the matrix includes a natural fiber.

3. s The substance according to claim 2, wherein the natural fiber is a coconut fiber.

4. The substance according to claim 2, wherein the natural fiber is an avocado fiber.

5. The substance according to claim 2, wherein the natural fiber is from a citrus fruit.

6. The substance according to claim 1, wherein the first part is a liquid.

7. The substance according to claim 1, wherein the first part is a solid.

8. The substance according to claim 1, wherein the second part is a liquid.

9. The substance according to claim 1, wherein the second part is a solid.

10. The substance wherein a weight of the first part is 1 milligram to a second weight of the second part of 1 milligram.

11. The substance wherein a weight of the first part is 1 milligram to a second weight of the second part of 2 milligrams.

12. The substance wherein a weight of the first part is 1 milligram to a second weight of the second part of 3 milligrams.

13. The substance wherein a weight of the first part is 1 milligram to a second weight of the second part of 4 milligrams.

14. The substance wherein a weight of the first part is 1 milligram to a second weight of the second part of 5 milligrams.

15. The substance wherein a weight of the first part is 1 milligram to a second weight of the second part of 6 milligrams.

16. The substance wherein a weight of the first part is 3 milligrams to a second weight of the second part of 10 milligrams.
